(19) United States
(12) Patent Application Publication
(10) Pub. No.: US 2024/0108636 A1
(43) Pub. Date: Apr. 4, 2024

(54) COMPOSITIONS FOR TREATMENT OF NEURODEGENERATIVE CONDITIONS

(71) Applicant: BIOVIE INC., Carson City, NV (US)

(72) Inventors: Clarence Nathaniel Ahlem, San Diego, CA (US); Christopher L. Reading, San Diego, CA (US)

(21) Appl. No.: 18/511,027

(22) Filed: Nov. 16, 2023

Related U.S. Application Data
(63) Continuation of application No. PCT/US2022/027294, filed on May 2, 2022.
(60) Provisional application No. 63/189,880, filed on May 18, 2021.

(51) Int. Cl. A61K 31/565; A61K 31/13; A61P 25/16

(52) U.S. Cl. CPC A61K 31/565; A61K 31/13; A61P 25/16

(57) ABSTRACT

Disclosed herein is a pharmaceutical composition comprising solid state forms of 17-ethynyl-10R, 13S-dimethyl 2, 3, 4, 7, 8R, 9S, 10, 11, 12, 13, 14S, 15, 16, 17-hexadecahydro-1H-cyclopenta[a]phenanthrene-3R, 7R, 17S-triol. Also disclosed herein is a method for using the solid state forms for the treatment of conditions related to neurodegenerative conditions including Parkinson's disease.

### INCORPORATION BY REFERENCE TO ANY PRIORITY APPLICATIONS

This application is a continuation of PCT App. No. PCT/US2022/027294, filed on May 2, 2022, which claims the priority benefit of U.S. Provisional App. No. 63/189,880, filed on May 18, 2021, which is hereby incorporated by reference in its entirety.

### BACKGROUND

### Field of the Disclosure

The present disclosure relates generally to the compound 17-ethynyl-10R, 13S-dimethyl 2, 3, 4, 7, 8R, 9S, 10, 11, 12, 13, 14S, 15, 16, 17-hexadecahydro-1H-cyclopenta[a]phenanthrene-3R, 7R, 17S-triol, solid state forms of the compound and methods for using the compound and the solid state forms.

The present disclosure relates to pharmaceutical compositions of the compound and the solid state forms and to methods for using the compound and the solid state forms, including the polymorph forms and pseudopolymorph forms, in preparing solid and liquid formulations.

The present disclosure relates to methods for using the compound, the solid state forms, the pharmaceutical compositions and the solid and liquid formulations for the treatment of conditions related to neurodegenerative conditions including Parkinson's disease.

### Description of the Related Art

Levodopa (L-3,4-dihydroxyphenylalanine), also known as L-dopa, is the most effective treatment for alleviating the motor symptoms caused by Parkinson's disease (PD), but it does not slow disease progression. Levodopa therapy relies on active transport of exogenous levodopa by the large neutral amino acid transporter into the central nervous system (CNS) where it is decarboxylated by aromatic L-amino acid decarboxylase to yield dopamine. Critically, dopamine cannot cross the blood-brain barrier to enter the CNS such that metabolization of exogenous levodopa in the periphery creates unusable dopamine in the bloodstream. Exogenous levodopa has a short half-life of about 90 minutes and there is limited capacity for storage of dopamine in dopaminergic terminals in the CNS so that levodopa must be administered frequently to be effective in alleviating the motor symptoms of PD.

When levodopa therapy is first instituted, PD patients typically experience a smooth and prolonged response with an absence of motor symptoms and few side effects. However, as PD progresses, degeneration of dopaminergic neurons leads to less efficient storage and release of levodopa and beneficial effects begin to wear off three to four hours after dosage. Such reduction in efficacy before the next scheduled dose is termed “wearing off”. As larger dosages of levodopa are administered various side effects such as motor complications frequently emerge over time. Motor complications associated with levodopa therapy occur in 30 to 40 percent of patients during the first five years of use and in at least 60 percent of patients by 10 years.

Levodopa combination therapy administers a second medicament that slows levodopa metabolization in the periphery and allows administration of less levodopa with the benefit of a delay in the onset of motor complications. One class of medicaments used for levodopa combination therapy are decarboxylase inhibitors that cannot cross the blood-brain barrier such as carbidopa (L-alpha-(3,4-dihydroxybenzyl)-alpha-hydrazinopropionic acid). However, levodopa carbidopa combination therapy includes numerous side effects that can include dizziness, loss of appetite, diarrhea, dry mouth, mouth and throat pain, constipation, change in sense of taste, forgetfulness, or confusion.

Levodopa metabolism increases oxidative stress in dopaminergic neurons, and it has been suggested that this increases the rate of PD progression. However, recent publications indicate there is no definitive evidence for disease acceleration by levodopa. In a majority of Parkinson's patients, long term pulsatile levodopa exposure produces adaptive responses in dopaminergic and serotonergic neurons and other adaptations that result in hyper-sensitivity to dopamine and involuntary abnormal movements called levodopa-induced dyskinesia (LID). The pathophysiology of LID is complex and manifestations can vary between patients relative to levodopa pharmacodynamics. Increased LID severity as a function of time is common, and LID expression can become as troublesome to patients as Parkinson's disease itself. Inflammatory extracellular signal regulated kinases 1 and 2 (ERK1/2, ERK) have been linked to LID expression in animal models, but specific mechanisms linking inflammation to LID have not been completely elucidated. Although anti-inflammatory strategies have been reported to reduce LID in rodent models, analogous publications of nonhuman primate models are lacking.

Various dopamine receptor agonists and inhibitors of dopamine metabolism (monoamine oxidase inhibitors, MAOIs) have been developed that provide motoric therapy with less potential to promote LID. These levodopa alternatives provide important options that allow patients to delay the start of levodopa, which for most patients starts a predictable path to LID, although a recent review casts doubt on the association between levodopa exposure and time to LID. Unfortunately, many dopamine receptor agonists have undesirable side effects or less promotoric activity than levodopa, whereas the promotor activity of MAOIs is useful as a monotherapy early in the disease only.

Efforts to develop therapies for LID have been largely based on selective dopamine receptor antagonism and non-pulsatile levodopa administration strategies. Neuropharmacologists working with medicinal chemists have developed highly selective dopamine receptor antagonists that decrease LID expression but these therapies also generally decrease promotoric dopaminergic signaling. Strategies to reduce the pulsatile character of oral levodopa administration include slow, continuous release oral formulations and L-dopa continuous jejununal infusion, which has had the greatest utility in patients with unusually high LID susceptibility. Despite these treatment options, LID remains an unsolved problem in the general treatment paradigm for the majority of PD patients. The high probability of levodopa therapy inducing LID provides the rationale for development of non-dopaminergic therapeutic alternatives to levodopa.

The need exists for non-dopaminergic therapeutic compositions for the treatment of PD. The need also exists for a non-dopaminergic therapeutic composition for use in levodopa combination therapy that can decrease motor symptoms, slow the onset of motor complications and present fewer side effects. The need exists for a non-dopaminergic therapeutic compositions for the treatment of LID.

### SUMMARY

Disclosed herein are methods to treat a neurodegenerative condition, the methods comprising administering to a patient in need thereof an effective amount of pharmaceutical compositions comprising 17α-ethynylandrost-5-ene-3β,7β,17β-triol and at least one pharmaceutically acceptable excipient. In several embodiments, the neurodegenerative condition is Parkinson's disease.

In several embodiments, the methods further comprise administering at least one additional medicament to the patient. In several embodiments, the additional medicament comprises at least one dopamine agonist. In several embodiments, the additional medicament comprises at least one dopamine precursor. In several embodiments, the additional medicament comprises L-dopa.

In several embodiments, the additional medicament is administered at a delay time after a first administration of the composition. In several embodiments, the delay time is equal to or greater than 2 years. In some embodiments, the delay time is 2, 3, or 4 years. In some embodiments, the delay of time is 5 years. In some embodiments, the delay time is greater than 5 years.

In several embodiments, at least one motor symptom develops in the patient. In several embodiments, the motor symptom is selected from tremor and/or shaking in the extremities, slowed movement (bradykinesia), muscle stiffness, rigidity, immobility (freezing), muscle cramps, impaired posture and/or balance, falls, dizziness, loss of automatic movements such as blinking or smiling, changes in speech and/or writing, motor fluctuations, dystonia, and any combination of the foregoing.

In several embodiments, at least one motor complication develops in the patient. In several embodiments, the motor complication is selected from wearing off, dose failure, beginning of dose worsening, end-of-dose rebound, unpredictable off-periods, freezing of gait, on-period failure, acute akinesia, dyskinesia, and any combination of the foregoing.

In several embodiments, the motor symptom develops at a time equal to or greater than 2, 2.5, 3, 3.5, 4, 4.5 or 5 years after the additional medicament is administered.

In several embodiments, the motor complication develops at a time equal to or greater than 2, 2.5, 3, 3.5, 4, 4.5 or 5 years after the additional medicament is administered.

In several embodiments, the 17α-ethynylandrost-5-ene-3β,7β,17β-triol is a solid state form of 17α-ethynylandrost-5-ene-3β,7β,17β-triol. In several embodiments, the solid state form of 17α-ethynylandrost-5-ene-3β,7β,17β-triol is crystalline solvate of 17α-ethynylandrost-5-ene-3β,7β,17β-triol. In several embodiments, the crystalline solvate is crystalline methanolate 17α-ethynylandrost-5-ene-3β,7β,17β-triol. In several embodiments, the crystalline solvate is crystalline ethanolate 17α-ethynylandrost-5-ene-3β,7β,17β-triol. In several embodiments, the crystalline solvate is crystalline hydrate 17α-ethynylandrost-5-ene-3β,7β,17β-triol. In several embodiments, the crystalline solvate is Form III 17α-ethynylandrost-5-ene-3β,7β,17β-triol. In several embodiments, the crystalline solvate is Form IV 17α-ethynylandrost-5-ene-3β,7β,17β-triol. In several embodiments, the crystalline solvate is Form V 17α-ethynylandrost-5-ene-3β,7β,17β-triol. In several embodiments, the solid state form of 17α-ethynylandrost-5-ene-3β,7β,17β-triol is amorphous 17α-ethynylandrost-5-ene-3β,7β,17β-triol.

In several embodiments, the pharmaceutical compositions contain less than about 3% by weight of impurities.

In several embodiments, the patient is a human or mammal.

Also disclosed herein are pharmaceutical compositions including 17α-ethynylandrost-5-ene-3β,7β,17β-triol for use in treating a neurodegenerative condition. In several embodiments, the pharmaceutical compositions include at least one pharmaceutically acceptable excipient. In several embodiments, the neurodegenerative condition is Parkinson's disease.

Also disclosed herein is the use of pharmaceutical compositions including 17α-ethynylandrost-5-ene-3β,7β,17β-triol for treating a neurodegenerative condition. In several embodiments, the use includes at least one pharmaceutically acceptable excipient. In several embodiments, the neurodegenerative condition is Parkinson's disease.

Also disclosed herein is the use of 17α-ethynylandrost-5-ene-3β,7β,17β-triol in the manufacture of a medicament for treating a neurodegenerative condition. In several embodiments, the medicament includes at least one pharmaceutically acceptable excipient. In several embodiments, the neurodegenerative condition is Parkinson's disease.

### BRIEF DESCRIPTION OF THE DRAWINGS

FIG. 1 illustrates immobility scores of test subjects treated with a pharmaceutical composition in accordance with an embodiment of the present disclosure.

FIG. 2 illustrates abnormal involuntary movements scores of test subjects treated with a pharmaceutical composition in accordance with an embodiment of the present disclosure.

FIG. 3 illustrates the percentage of surviving neurons of test subjects treated with a pharmaceutical composition in accordance with an embodiment of the present disclosure.

FIG. 4 illustrates body weight of test subjects treated with a pharmaceutical composition in accordance with an embodiment of the present disclosure.

### DETAILED DESCRIPTION

The following description provides context and examples, but should not be interpreted to limit the scope of the disclosure covered by the claims that follow in this specification or in any other application that claims priority to this specification. No single component or collection of components is essential or indispensable. For example, in some embodiments one or more variables, such as Y or Y and Q may be omitted. Any feature, structure, component, material, step, or method that is described and/or illustrated in any embodiment in this specification can be used with or instead of any feature, structure, component, material, step, or method that is described and/or illustrated in any other embodiment in this specification.

### Definitions

The term “dopamine agonist,” as used herein, is a substance or medicament hat can mimic the actions of dopamine when ingested. These substances can improve the symptoms that are related to insufficient levels of dopamine in a subject.

The term “dopamine precursor,” as used herein, is a substance or medicament that is converted into dopamine within the body. These substances can enter the brain and restore depleted levels of dopamine.

As used herein, “subject,” “host,” “patient,” and “individual” are used interchangeably and shall be given their ordinary meaning in the art and shall also refer to an organism that has cancer and/or leukemia. This includes mammals, e.g., a human, a non-human primate, ungulates, canines, felines, equines, mice, rats, and the like. The term “mammal” includes both human and non-human mammals.

The terms “therapeutically effective amount” and “effective amount” refer to the amount of active pharmaceutical ingredient necessary to provide the desired pharmacologic result. In practice, the therapeutically effective amount will vary widely depending on the severity of the disease condition, age of the subject, and the desired therapeutic effect.

The terms “treatment,” “treating,” “treat,” and the like shall be given their ordinary meaning and shall also include herein to generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. The terms “treatment,” as used herein shall be given its ordinary meaning and shall also cover any treatment of a disease in a mammal, particularly a human, and includes: (a) preventing the disease or symptom from occurring in a subject which may be predisposed to the disease or symptom but has not yet been diagnosed as having it; (b) inhibiting the disease symptom, e.g., arresting its development; and/or (c) relieving the disease symptom, e.g., causing regression of the disease or symptom.

All literature and similar materials cited in this application, including but not limited to, patents, patent applications, articles, books, treatises, and internet web pages are expressly incorporated by reference in their entirety for any purpose. When definitions of terms in incorporated references appear to differ from the definitions provided in the present teachings, the definition provided in the present teachings shall control. It will be appreciated that there is an implied “about” prior to the temperatures, concentrations, times, etc. discussed in the present teachings, such that slight and insubstantial deviations are within the scope of the present teachings herein. In this application, the use of the singular includes the plural unless specifically stated otherwise. Also, the use of “comprise”, “comprises”, “comprising”, “contain”, “contains”, “containing”, “include”, “includes”, and “including” are not intended to be limiting. It is to be understood that both the general description and the following detailed description are exemplary and explanatory only and are not restrictive. The term “and/or” denotes that the provided possibilities can be used together or be used in the alternative. Thus, the term “and/or” denotes that both options exist for that set of possibilities.

Terms and phrases used in this application, and variations thereof, especially in the appended claims, unless otherwise expressly stated, should be construed as open ended as opposed to limiting. As examples of the foregoing, the term “including” should be read to mean “including, without limitation,” “including but not limited to,” or the like; the term “comprising” as used herein is synonymous with “including,” “containing,” or “characterized by,” and is inclusive or open-ended and does not exclude additional, unrecited elements or method steps; the term “having” should be interpreted as “having at least;” the term “includes” should be interpreted as “includes but is not limited to;” the term “example” is used to provide exemplary instances of the item in discussion, not an exhaustive or limiting list thereof; and use of terms like “preferably,” “preferred,” “desired,” or “desirable,” and words of similar meaning should not be understood as implying that certain features are critical, essential, or even important to the structure or function of the invention, but instead as merely intended to highlight alternative or additional features that may or may not be utilized in a particular embodiment of the disclosure. In addition, the term “comprising” is to be interpreted synonymously with the phrases “having at least” or “including at least”. When used in the context of a process, the term “comprising” means that the process includes at least the recited steps, but may include additional steps. When used in the context of a compound, composition or device, the term “comprising” means that the compound, composition or device includes at least the recited features or components, but may also include additional features or components. Likewise, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should be read as “and/or” unless expressly stated otherwise.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

### Methods of Treatment

Embodiments of the present disclosure relate to methods to treat a neurodegenerative condition. In several embodiments, the methods include administering to a patient in need thereof an effective amount of a pharmaceutical composition. In several embodiments, the pharmaceutical compositions include 17-ethynyl-10R, 13S-dimethyl 2, 3, 4, 7, 8R, 9S, 10, 11, 12, 13, 14S, 15, 16, 17-hexadecahydro-1H-cyclopenta[a]phenanthrene-3R, 7R, 17S-triol, which is represented by Formula 1. The compound of Formula 1 is hereafter also referred to as Compound 1 or 17α-ethynylandrost-5-ene-3β,7β,17β-triol.

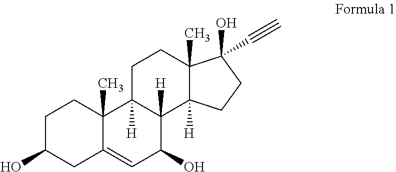

In several embodiments, the neurodegenerative condition is Alzheimer's disease, Parkinson's disease or Amyotrophic Lateral Sclerosis. In several embodiments, the neurodegenerative condition is Parkinson's disease, parkinsonisms, a parkinsonian syndrome, or any combination of the foregoing. In several embodiments, the neurodegenerative condition includes idiopathic Parkinson's disease, progressive supranuclear palsy, multiple system atrophy, corticobasal degeneration, and vascular parkinsonism, or any combination of the foregoing. In several embodiments, the neurodegenerative condition is Parkinson's disease.

In several embodiments, the patient is a human or mammal. In several embodiments, the patient is a human.

In several embodiments, the methods delay administering to the patient one or more dopamine agonists. In several embodiments, the methods delay administering to the patient L-dopa.

In several embodiments, the methods delay development of motor complications in the patient caused by one or more dopamine agonists. In several embodiments, the methods delay development of motor complications in the patient caused by L-dopa.

In several embodiments, the methods include administering at least one additional medicament to the patient. In several embodiments, the additional medicament comprises at least one dopamine agonist. In several embodiments, the additional medicament comprises an ergoline dopamine agonist, a non-ergoline dopamine agonist, or any combination of the foregoing. Non-limiting examples of suitable dopamine agonists include bromocriptine, cabergoline, apomorphine, pramipexole, ropinirole, rotigotine, APOKYN, KYNMOBI, MIRAPEX, NEUPRO, PARLODEL, and REQUIP. In several embodiments, the additional medicament comprises at least one dopamine precursor. Non-limiting examples of suitable dopamine precursors include carbidopa, L-dopa (i.e., levodopa), entacapone, COMTAN, DUOPA, INBRIJA, RYTARY, SINEMET, SINEMET CR, and STALEVO. In several embodiments, the additional medicament comprises L-dopa.

In several embodiments, the additional medicament is administered at a delay time after a first administration of the composition. In several embodiments, the first administration may occur using a dosage schedule that is daily, weekly, monthly, or any combination of the foregoing. In several embodiments, the dosage schedule of the first administration may include one, two, three or more daily dosages of the composition. In several embodiments, the dosage schedule of the first administration may include one, two, three or more weekly dosages of the composition. In several embodiments, the dosage schedule of the first administration may include one, two, three or more monthly dosages of the composition. In several embodiments, the delay time is equal to or greater than about: 0.5, 1, 1.5, 2, 3, 4, 5, 6, 7, 8, 9, 10, 11, 12, 13, 14, 15, 16, 17, 18, 19, 20 years, or ranges including and/or spanning the aforementioned values. In several embodiments, the delay time is equal to or greater than 2 years. In some embodiments, the delay time is zero and the additional medicament is administered concurrently with the first administration of the composition. In several embodiments, the additional medicament is administered using a dosage schedule that is daily, weekly, monthly, or any combination of the foregoing. In several embodiments, the dosage schedule of the additional medicament may include one, two, three or more daily dosages of the composition. In several embodiments, the dosage schedule of the additional medicament may include one, two, three or more weekly dosages of the composition. In several embodiments, the dosage schedule of the additional medicament may include one, two, three or more monthly dosages of the composition.

In several embodiments, at least one motor symptom develops in the patient. The term “motor symptom,” as used herein, is one or more of tremor and/or shaking in the extremities, slowed movement (bradykinesia), muscle stiffness, rigidity, immobility (freezing), muscle cramps, impaired posture and/or balance, falls, dizziness, loss of automatic movements such as blinking or smiling, changes in speech and/or writing, motor fluctuations, and dystonia. The term “dystonia,” as used herein, is a state of abnormal muscle tone resulting in muscular spasm and abnormal posture.

In several embodiments, at least one motor complication develops in the patient. The term “motor complication,” as used herein, can be a cycling between on-periods and off-periods. The term “on-period,” as used herein, is a state of positive response to the composition, the additional medicament, or a combination thereof. The on-period is characterized by the absence of, or a decrease in, symptoms related to Parkinson's disease, parkinsonisms, a parkinsonian syndrome, or any combination of the foregoing. The term “off-period,” as used herein, is a state of no response to the composition, the additional medicament, or a combination thereof. The off-period is characterized by the presence of symptoms related to Parkinson's disease, parkinsonisms, a parkinsonian syndrome, or any combination of the foregoing. In several embodiments, the symptoms related to Parkinson's disease parkinsonisms, and/or a parkinsonian syndrome may include at least one motor symptom, as described elsewhere herein.

In several embodiments, the motor complications can include wearing off, dose failure, beginning of dose worsening, end-of-dose rebound, unpredictable off-periods, freezing of gait, on-period failure, acute akinesia, or a combination of the foregoing. The term “wearing off,” as used herein, is a reemergence of symptoms related to Parkinson's disease that occurs as metabolization of the composition, the additional medicament, or a combination thereof reaches completion. The term “unpredictable off-period,” is an off-period that is not correlated with the administration the composition, the additional medicament, or a combination thereof. The term “freezing of gait,” as used herein, is a state in which forward progression halts or is markedly reduced. The term “on-period failure,” as used herein, is absence of an on-period after the administration the composition, the additional medicament, or a combination thereof. The term “acute akinesia,” as used herein, is a sudden and/or severe increase of symptoms related to Parkinson's disease including an akinetic state that lasts for several days and does not respond to the administration the composition, the additional medicament, or a combination thereof.

In several embodiments, the motor complications can include dyskinesia. The term “dyskinesia,” as used herein, is an abnormality or impairment of voluntary movement and/or posture. In several embodiments, the motor complications include dyskinesia selected from L-dopa-induced dyskinesia, chorea, dystonia, ballism, myoclonus, peak-dose dyskinesia, diphasic dyskinesia, wearing-off dyskinesia, wearing-off dystonia, or a combination of the foregoing. The term “peak-dose dyskinesia,” as used herein, appears during an on-period and begins at about 30 to 90 minutes after administration the composition, the additional medicament, or a combination thereof. The term “diphasic dyskinesia,” as used herein, includes two separate periods of involuntary movement after administration the composition, the additional medicament, or a combination thereof. The first occurring at the start of an on-period and the second occurring at the start of an off-period. The term “wearing-off dyskinesia,” as used herein, is less common and sometimes characterized by large-amplitude leg movements. The term “wearing-off dystonia,” as used herein, manifests as dystonia during an off-periods and usually involves the limbs but sometimes involving the face, neck, or trunk. In several embodiments, the dyskinesia appears at various times in relation to the administration the composition, the additional medicament, or a combination thereof.

In several embodiments, the motor symptom occurs when the composition, the additional medicament, or a combination thereof has been completely metabolized. In several embodiments, the motor symptom occurs when the composition has been completely metabolized. In several embodiments, the motor symptom occurs when the additional medicament has been completely metabolized. In several embodiments, the motor symptom occurs when the dopamine agonist has been completely metabolized. In several embodiments, the motor symptom occurs when the dopamine precursor has been completely metabolized. In several embodiments, the motor symptom occurs when the L-dopa has been completely metabolized.

In several embodiments, the motor complication occurs when the composition, the additional medicament, or a combination thereof has been completely metabolized. In several embodiments, the motor complication occurs when the composition has been completely metabolized. In several embodiments, the motor complication occurs when the additional medicament has been completely metabolized. In several embodiments, the motor complication occurs when the dopamine agonist has been completely metabolized. In several embodiments, the motor complication occurs when the dopamine precursor has been completely metabolized. In several embodiments, the motor complication occurs when the L-dopa has been completely metabolized.

In several embodiments, the motor symptom develops at a time equal to or greater than about 0.5, 1, 1.5, 2, 3, 4, 5, 6, 7, 8, 9, 10, 11, 12, 13, 14, 15, 16, 17, 18, 19, 20, or 30 years after the composition is administered. In several embodiments, the motor symptom develops at a time equal to or greater than about 0.5, 1, 1.5, 2, 3, 4, 5, 6, 7, 8, 9, 10, 11, 12, 13, 14, 15, 16, 17, 18, 19, 20, or 30 years after the additional medicament is administered. In several embodiments, the motor symptom develops at a time equal to or greater than about 0.5, 1, 1.5, 2, 3, 4, 5, 6, 7, 8, 9, 10, 11, 12, 13, 14, 15, 16, 17, 18, 19, 20, or 30 years after the dopamine precursor is administered. In several embodiments, the motor symptom develops at a time equal to or greater than about 0.5, 1, 1.5, 2, 3, 4, 5, 6, 7, 8, 9, 10, 11, 12, 13, 14, 15, 16, 17, 18, 19, 20, or 30 years after the L-dopa is administered.

In several embodiments, the motor complication develops at a time equal to or greater than about 0.5, 1, 1.5, 2, 3, 4, 5, 6, 7, 8, 9, 10, 11, 12, 13, 14, 15, 16, 17, 18, 19, 20, or 30 years after the composition is administered. In several embodiments, the motor complication develops at a time equal to or greater than about 0.5, 1, 1.5, 2, 3, 4, 5, 6, 7, 8, 9, 10, 11, 12, 13, 14, 15, 16, 17, 18, 19, 20, or 30 years after the additional medicament is administered. In several embodiments, the motor complication develops at a time equal to or greater than about 0.5, 1, 1.5, 2, 3, 4, 5, 6, 7, 8, 9, 10, 11, 12, 13, 14, 15, 16, 17, 18, 19, 20, or 30 years after the dopamine precursor is administered. In several embodiments, the motor complication develops at a time equal to or greater than about 0.5, 1, 1.5, 2, 3, 4, 5, 6, 7, 8, 9, 10, 11, 12, 13, 14, 15, 16, 17, 18, 19, 20, or 30 years after the L-dopa is administered.

In several embodiments, the pharmaceutical compositions include a solid state form of 17α-ethynylandrost-5-ene-3β,7β,17β-triol. In several embodiments, the solid state form is crystalline 17α-ethynylandrost-5-ene-3β,7β,17β-triol. In several embodiments, the solid state form is crystalline 17α-ethynylandrost-5-ene-3β,7β,17β-triol substantially free of 17α-ethynylandrost-5-ene-3β,7β,17β-triol in amorphous form.

In several embodiments, the solid state form is crystalline solvate 17α-ethynylandrost-5-ene-3β,7β,17β-triol. In several embodiments, the crystalline solvate is crystalline methanolate 17α-ethynylandrost-5-ene-3β,7β,17β-triol. In several embodiments, the crystalline solvate is crystalline ethanolate 17α-ethynylandrost-5-ene-3β,7β,17β-triol. In several embodiments, the crystalline solvate is crystalline hydrate 17α-ethynylandrost-5-ene-3β,7β,17β-triol.

In several embodiments, the crystalline solvate is Form III 17α-ethynylandrost-5-ene-3β,7β,17β-triol. In several embodiments, the crystalline solvate is Form IV 17α-ethynylandrost-5-ene-3β,7β,17β-triol. In several embodiments, the crystalline solvate is Form V 17α-ethynylandrost-5-ene-3β,7β,17β-triol.

In several embodiments, the solid state form of 17α-ethynylandrost-5-ene-3β,7β,17β-triol is amorphous 17α-ethynylandrost-5-ene-3β,7β,17β-triol. In several embodiments, the amorphous 17α-ethynylandrost-5-ene-3β,7β,17β-triol substantially free of 17α-ethynylandrost-5-ene-3β,7β,17β-triol in solid state form.

In several embodiments, the pharmaceutical compositions include at least one pharmaceutically acceptable excipient. Non-limiting examples of pharmaceutically acceptable excipients suitable for use in the compositions include fillers, diluents, disintegrants, binders, glidants, and/or lubricants. Other pharmaceutically acceptable excipients suitable for use in the compositions include absorption enhancing agents, acidifying agents, agents for modified release, alkalizing agents, antioxidants, buffering agents, chelating agents, coloring agents, complexing agents, emulsifying agents, flavoring agents, humectants, humidity-adjusting agents, pH-adjusting agents, preservatives, solubilizing agents, stabilizers, surface-active agents, suspending agents, sweetening agents, taste-masking agents, and wetting agents.

Non-limiting examples of fillers suitable for use in the compositions include lactose, microcrystalline cellulose, hydroxypropylcellulose, hydroxypropyl methylcellulose, methyl cellulose polymers hydroxyethylcellulose, sodium carboxymethylcellulose, carboxymethylene, carboxymethylhydroxyethylcellulose and other cellulose derivatives, sucrose, agarose, sorbitol, mannitol, dextrins, maltodextrins, starches or modified starches (including potato starch, maize starch and rice starch), calcium phosphate (e.g. basic calcium phosphate, calcium hydrogen phosphate, dicalcium phosphate hydrate), calcium sulfate, calcium carbonate, sodium alginate, and collagen.

Non-limiting examples of diluents suitable for use in the compositions include e.g. calcium carbonate, dibasic calcium phosphate, tribasic calcium phosphate, calcium sulfate, microcrystalline cellulose, powdered cellulose, dextrans, dextrin, dextrose, fructose, kaolin, lactose, mannitol, sorbitol, starch, pregelatinized starch, sucrose, and sugar.

Non-limiting examples of disintegrants suitable for use in the compositions include alginic acid or alginates, microcrystalline cellulose, low-substituted hydroxypropyl cellulose and other cellulose derivatives, croscarmellose sodium, crospovidone, polacrillin potassium, sodium starch glycolate, starch, pregelatinized starch, and carboxymethyl starch.

Non-limiting examples of binders suitable for use in the compositions include acacia, alginic acid, agar, calcium carrageenan, sodium carboxymethylcellulose, microcrystalline cellulose, dextrin, ethylcellulose, gelatin, liquid glucose, guar gum, hydroxypropyl methylcellulose, methylcellulose, pectin, PEG, polyethylene oxides, povidone, and pregelatinized starch.

Non-limiting examples of glidants and/or lubricants suitable for use in the compositions include stearic acid, magnesium stearate, calcium stearate or other metallic stearates, talc, waxes and glycerides, light mineral oil, PEG, glyceryl behenate, colloidal silica, hydrogenated vegetable oils, corn starch, sodium stearyl fumarate, polyethylene glycols, alkyl sulfates, sodium benzoate, and sodium acetate.

Non-limiting examples of antioxidants suitable for use in the compositions include ascorbic acid, ascorbyl palmitate, butylated hydroxyanisole, butylated hydroxytoluene, hypophosphorous acid, monothioglycerol, potassium metabisulfite, propyl gallate, sodium formaldehylde sulfoxylate, sodium metabisulfite, sodium thiosulfate, sulfur dioxide, tocopherol, tocopherol acetate, tocopherol hemisuccinate, and derivatives of tocopherol.

In several embodiments, the pharmaceutically acceptable excipient is selected from sodium dodecyl sulfate, microcrystalline cellulose, magnesium stearate, and any combination of the foregoing. In several embodiments, the pharmaceutically acceptable excipient is sodium dodecyl sulfate.

In several embodiments, the pharmaceutical compositions are formulated into oral dosage forms. In several embodiments, the dosage forms can include capsules and tablets. In some embodiments, the dosage forms can include one or more different types of delayed release layers selected from sealant and/or enteric layers. For example, delayed release layers having different release rate characteristics can provide the dosage form with different overall drug release characteristics. In some such embodiments, the pharmaceutically acceptable excipient is a surface active agent. In several embodiments, the surface active agent is present in an amount sufficient to provide 90% dissolution of the pharmaceutical composition in water at ambient temperature after 30 min. In several embodiments, the surface active agent is sodium lauryl sulfate. In several embodiments, the pharmaceutical composition is a capsule or a tablet.

In several embodiments, the pharmaceutical compositions contain less than about 3% by weight of impurities.

In several embodiments, the pharmaceutical compositions include a pharmaceutically acceptable formulation of 17α-ethynylandrost-5-ene-3β,7β,17β-triol.

Several embodiments of the present disclosure relate to pharmaceutical compositions including 17α-ethynylandrost-5-ene-3β,7β,17β-triol for use in treating a neurodegenerative condition. In several embodiments, the neurodegenerative condition is Alzheimer's disease, Parkinson's disease or Amyotrophic Lateral Sclerosis. In several embodiments, the neurodegenerative condition is Parkinson's disease, parkinsonisms, a parkinsonian syndrome, or any combination of the foregoing. In several embodiments, the neurodegenerative condition includes idiopathic Parkinson disease, progressive supranuclear palsy, multiple system atrophy, corticobasal degeneration, and vascular parkinsonism, or any combination of the foregoing. In several embodiments, the neurodegenerative condition is Parkinson's disease. In several embodiments, the pharmaceutical compositions include at least one pharmaceutically acceptable excipient.

In several embodiments, the pharmaceutical compositions are used with at least one additional medicament. In several embodiments, the additional medicament includes at least one dopamine agonist, as described elsewhere herein. In several embodiments, the additional medicament includes at least one dopamine precursor as described elsewhere herein. In several embodiments, the additional medicament includes L-dopa. In several embodiments, the additional medicament is used at a delay time after use of the pharmaceutical composition begins. In several embodiments, the delay time is equal to or greater than 0.5, 1, 1.5, 2, 3, 4, 5, 6, 7, 8, 9, 10, 11, 12, 13, 14, 15, 16, 17, 18, 19, 20 years, or ranges including and/or spanning the aforementioned values. In several embodiments, the delay time is equal to or greater than 2 years.

In several embodiments, at least one motor symptom can develop while the pharmaceutical compositions are used. Such motor symptoms are described elsewhere herein. In several embodiments, the motor symptom develops at a time after the use of the pharmaceutical composition begins that is 0.5, 1, 1.5, 2, 3, 4, 5, 6, 7, 8, 9, 10, 11, 12, 13, 14, 15, 16, 17, 18, 19, 20 years, or ranges including and/or spanning the aforementioned values. In several embodiments, the motor symptom develops at a time equal to or greater than 2 years after the use of the pharmaceutical composition begins. In several embodiments, the motor symptom develops at a time after the use of the additional medicament begins that is 0.5, 1, 1.5, 2, 3, 4, 5, 6, 7, 8, 9, 10, 11, 12, 13, 14, 15, 16, 17, 18, 19, 20 years, or ranges including and/or spanning the aforementioned values. In several embodiments, the motor symptom develops at a time equal to or greater than 2 years after the use of the additional medicament begins.

In several embodiments, at least one motor complication can develop while the pharmaceutical compositions are used. Such motor complications are described elsewhere herein. In several embodiments, the motor complication develops at a time after the use of the pharmaceutical composition begins that is 0.5, 1, 1.5, 2, 3, 4, 5, 6, 7, 8, 9, 10, 11, 12, 13, 14, 15, 16, 17, 18, 19, 20 years, or ranges including and/or spanning the aforementioned values. In several embodiments, the motor complication develops at a time equal to or greater than 2 years after the use of the pharmaceutical composition begins. In several embodiments, the motor complication develops at a time after the use of the additional medicament begins that is 0.5, 1, 1.5, 2, 3, 4, 5, 6, 7, 8, 9, 10, 11, 12, 13, 14, 15, 16, 17, 18, 19, 20 years, or ranges including and/or spanning the aforementioned values. In several embodiments, the motor complication develops at a time equal to or greater than 2 years after the use of the additional medicament begins.

Several embodiments of the present disclosure relate to the use of pharmaceutical compositions including 17α-ethynylandrost-5-ene-3β,7β,17β-triol for treating a neurodegenerative condition. In several embodiments, the neurodegenerative condition is Alzheimer's disease, Parkinson's disease or Amyotrophic Lateral Sclerosis. In several embodiments, the neurodegenerative condition is Parkinson's disease, parkinsonisms, a parkinsonian syndrome, or any combination of the foregoing. In several embodiments, the neurodegenerative condition includes idiopathic Parkinson disease, progressive supranuclear palsy, multiple system atrophy, corticobasal degeneration, and vascular parkinsonism, or any combination of the foregoing. In several embodiments, the neurodegenerative condition is Parkinson's disease. In several embodiments, the pharmaceutical compositions include at least one pharmaceutically acceptable excipient.

In several embodiments, the use is concurrent with a use of at least one additional medicament. In several embodiments, the additional medicament includes at least one dopamine agonist, as described elsewhere herein. In several embodiments, the additional medicament includes at least one dopamine precursor as described elsewhere herein. In several embodiments, the additional medicament includes L-dopa. In several embodiments, the use of the least one additional medicament includes a delay time after the use of the pharmaceutical composition begins. In several embodiments, the delay time is equal to or greater than 0.5, 1, 1.5, 2, 3, 4, 5, 6, 7, 8, 9, 10, 11, 12, 13, 14, 15, 16, 17, 18, 19, 20 years, or ranges including and/or spanning the aforementioned values. In several embodiments, the delay time is equal to or greater than 2 years. In several embodiments, the use includes development of at least one motor complication. Such motor complications are described elsewhere herein. In several embodiments, the motor complication develops at a time after the use of the pharmaceutical composition begins that is 0.5, 1, 1.5, 2, 3, 4, 5, 6, 7, 8, 9, 10, 11, 12, 13, 14, 15, 16, 17, 18, 19, 20 years, or ranges including and/or spanning the aforementioned values. In several embodiments, the motor complication develops at a time equal to or greater than 2 years after the use of the pharmaceutical composition begins. In several embodiments, the motor complication develops at a time after the use of the additional medicament begins that is 0.5, 1, 1.5, 2, 3, 4, 5, 6, 7, 8, 9, 10, 11, 12, 13, 14, 15, 16, 17, 18, 19, 20 years, or ranges including and/or spanning the aforementioned values. In several embodiments, the motor complication develops at a time equal to or greater than 2 years after the use of the additional medicament begins.

Several embodiments of the present disclosure relate to the use of 17α-ethynylandrost-5-ene-3β,7β,17β-triol in the manufacture of a medicament for treating a neurodegenerative condition. In several embodiments, the neurodegenerative condition is Alzheimer's disease, Parkinson's disease or Amyotrophic Lateral Sclerosis. In several embodiments, the neurodegenerative condition is Parkinson's disease, parkinsonisms, a parkinsonian syndrome, or any combination of the foregoing. In several embodiments, the neurodegenerative condition includes idiopathic Parkinson disease, progressive supranuclear palsy, multiple system atrophy, corticobasal degeneration, and vascular parkinsonism, or any combination of the foregoing. In several embodiments, the neurodegenerative condition is Parkinson's disease. In several embodiments, the medicament includes at least one pharmaceutically acceptable excipient.

In several embodiments, the medicament includes at least one additional medicament. In several embodiments, the additional medicament includes at least one dopamine agonist, as described elsewhere herein. In several embodiments, the additional medicament includes at least one dopamine precursor as described elsewhere herein. In several embodiments, the additional medicament includes L-dopa.

In several embodiments, the use includes a delay time after use of the medicament begins. In several embodiments, the delay time is equal to or greater than 0.5, 1, 1.5, 2, 3, 4, 5, 6, 7, 8, 9, 10, 11, 12, 13, 14, 15, 16, 17, 18, 19, 20 years, or ranges including and/or spanning the aforementioned values. In several embodiments, the delay time is equal to or greater than 2 years.

In several embodiments, the use includes development of at least one motor symptom. Such motor symptoms are described elsewhere herein. In several embodiments, the motor symptom develops at a time after the use of the medicament begins that is 0.5, 1, 1.5, 2, 3, 4, 5, 6, 7, 8, 9, 10, 11, 12, 13, 14, 15, 16, 17, 18, 19, 20 years, or ranges including and/or spanning the aforementioned values. In several embodiments, the motor symptom develops at a time equal to or greater than 2 years after the use of the medicament begins. In several embodiments, the motor symptom develops at a time after the use of the additional medicament begins that is 0.5, 1, 1.5, 2, 3, 4, 5, 6, 7, 8, 9, 10, 11, 12, 13, 14, 15, 16, 17, 18, 19, 20 years, or ranges including and/or spanning the aforementioned values. In several embodiments, the motor symptom develops at a time equal to or greater than 2 years after the use of the additional medicament begins.

In several embodiments, the use includes development of at least one motor complication. Such motor complications are described elsewhere herein. In several embodiments, the motor complication develops at a time after the use of the medicament begins that is 0.5, 1, 1.5, 2, 3, 4, 5, 6, 7, 8, 9, 10, 11, 12, 13, 14, 15, 16, 17, 18, 19, 20 years, or ranges including and/or spanning the aforementioned values. In several embodiments, the motor complication develops at a time equal to or greater than 2 years after the use of the medicament begins. In several embodiments, the motor complication develops at a time after the use of the additional medicament begins that is 0.5, 1, 1.5, 2, 3, 4, 5, 6, 7, 8, 9, 10, 11, 12, 13, 14, 15, 16, 17, 18, 19, 20 years, or ranges including and/or spanning the aforementioned values. In several embodiments, the motor complication develops at a time equal to or greater than 2 years after the use of the additional medicament begins.

The subject matter of the present application includes features discussed in U.S. Pat. No. 8,252,947, issued Aug. 28, 2012, which is hereby incorporated by reference in its entirety for all purposes.

### EXAMPLES

### Example 1. Inflammatory Pathways of Neurodegenerative Mechanisms Linked to Pd Progression

The great majority of Parkinson's disease (PD) is characterized as idiopathic and a minority have a known genetic basis most frequently linked to mutations in the neuronal protein, alpha-synuclein (SNCA) and genes associated with mitochondrial homeostasis. Over-production, misfolding, and aggregation of SNCA is a major contributor to neuronal oxidative stress and energy dyshomeostasis. Misfolded SNCA acts as a prion and is believed to have a role in disease spreading within the central nervous system (CNS), and there is evidence of transmission from the gut to the CNS as a possible means of disease initiation. A widely held view is that motor symptoms of the disease result primarily from low levels of the neurotransmitter, dopamine, secondary to the loss of dopaminergic neurons in the substantia nigra. This dopamine-centric view tends to understate the critical contribution of inflammation to disease expression. Although a threshold of 50%-80% reduction in dopamine levels is generally assumed necessary to cause motoric symptoms parkinsonian behavior can be observed with considerably less neurodegeneration in animal models of PD initiated with neuroinflammatory agents. Moreover, reduction of neuroinflammation and oxidative stress without dopaminergic therapy can improve mobility and decrease clinical signs of disease in animal models and humans. Important to the concept of anti-inflammatory therapy of PD is pharmaceutical acceptability, which extends to drug candidate safety for chronic use, blood-brain barrier permeability, and a mechanism of action that targets critical aspects of the inflammatory processes driving disease expression and progression.

Inflammation and oxidative stress are mutually inductive and drive pathophysiology in neurodegenerative diseases. In PD, misfolded over-expressed aggregated SNCA interacts with molecular pattern receptors, toll-like receptor 4 (TLR4), and the receptor for advanced glycation end products (RAGE), to activate inflammatory signaling cascades controlled by specific extracellular signal-regulated kinase-nuclear factor-kappa B (ERK-NFkB)-containing scaffolds that mediate tumor necrosis factor (TNF), interleukin 1b (IL-1b), interleukin-6 (IL-6), and other inflammatory cytokine production. These inflammatory signaling mechanisms are independent from NFkB-ERK homeostatic signaling pathways that control cell proliferation, long-term potentiation, and insulin signaling (such as Ras/Raf/MEK/ERK).

Activation of inflammatory pathways triggers inducible nitric oxide synthase (iNOS) synthesis that promotes formation of reactive nitrogen and oxygen species that impact mitochondrial cytochrome efficiency to decrease energy production, increase calcium currents, and generate more reactive oxygen species. These oxidation species activate NFkB and calmodulin kinase in a cycle that tends to feed forward, creating a state of chronic inflammation and oxidative stress. Mitochondrial dysfunction in various forms is a primary driver of PD pathophysiology. Activated microglia and astrocytes play a prominent role in PD pathology and progression through maintenance of an inflammatory milieu. Accumulating evidence demonstrates the reactive oxygen species (ROS) and pro-inflammatory cytokines produced by microglia are engaged in the induction and perpetuation of the neurodegenerative processes in PD.

High energy demands of dopaminergic neurons make them especially vulnerable to deleterious effects of mitochondrial dysfunction. Maintenance of protein homeostasis is an energy intensive process. Endoplasmic reticulum (ER) stress results when cells lack sufficient reducing power to properly guide newly synthesized protein folding, preserve function and prevent aggregation. Under conditions of ER stress, SNCA can be misfolded and aggregated into oligomeric sheets that are toxic to mitochondria in addition to activating molecular pattern receptors and inflammatory pathways.

Insulin signaling plays a vital role in neuronal energy homeostasis and neuron survival. Inflammatory activation of mitogen associated protein kinases (MAPK) can inhibit insulin signaling (induce insulin resistance) through phosphorylation of various serine residues on insulin receptor substrate 1 and 2 (IRS-1/2) that interfere with insulin receptor tyrosine phosphorylation or interactions of IRS-1/2 with the insulin receptor or other proteins in the signaling complex. Inflammation triggered by SNCA can thus contribute to insulin resistance that hinders mitochondrial function and promotes oxidative and ER stress and feeds forward to increased SNCA dyshomeostasis. Interestingly, intranasal insulin has promotoric activity in PD, and insulin resistance has been linked to PD cognitive symptoms as well. It is reported that 60%-80% of PD subjects have insulin resistance. Several anti-diabetic agents are in clinical evaluation for potential benefits to PD patients (clinicaltrials.gov: NCT04251585, NCT02953665, NCT04232969).

Inflammatory chemokines promote infiltrating lymphocytes and macrophages that contribute to the inflammatory milieu in PD. An adaptive immune response of T cells recognizing SNCA peptides contributes to the neurodegenerative process with reactive astrocytes potentially acting as antigen-presenting cells that may also facilitate the spread of SNCA aggregates. Therefore, infiltrating lymphocytes have the potential to contribute to PD pathophysiology through multiple mechanisms, and decreasing inflammatory cell infiltration may have a significant impact on disease.

ERK mediated neuroinflammation is causally associated with levodopa-induced dyskinesia (LID), as described elsewhere herein, and genetic and pharmacological modulation of ERK activation reduces LID in rodent models. Furthermore, inflammatory signals mediated by inflammatory NFkB-ERK signaling are linked to excitotoxicity and well-established neurodegenerative mechanisms linked to PD progression. This pathogenic effector mechanism in PD patients is recapitulated in a valid disease model, elicited by injection of 1-methyl-4-phenyl-1,2,3,6-tetrahydropyridine (MPTP) into marmoset monkeys (Callithrix jacchus). MPTP is a blood-brain permeable substrate for the dopamine transporter that is metabolized to the mitochondrial toxin, 1-methyl-4-phenylpyridinium (MPP*) by monoamine oxidase in astocytes and selectively taken up by neurons through monoamine transporters. MPTP administration to animals (and humans) produces Parkinson's-like symptoms that result from loss of dopaminergic cells (neurodegeneration) in the substantia nigra pars compacta (SNpc) and the consequent reduced striatal dopamine concentrations and neuroinflammation. Selective injury of dopaminergic (DA) cells after MPTP intoxication is immediately followed by the clustering of microglia around injured neurons. The reaction of microglia expressed myeloperoxidase (MPO) and H₂O₂ with structurally-related ortho-methoxy-substituted catechols, such as apocynin and vanillic acid, generates reactive intermediates that bind to free thiol groups. MPO is up regulated in activated brain microglia cells of PD patients and in the MPTP-induced animal model. As with Parkinson's disease (which is unique to humans) dopamine administration to MPTP-treated animals can significantly improve motor control, but does not decrease inflammatory mechanisms, and thus does not modify disease processes or slow progression in animal models. Therefore, selective inhibitors of microglia ROS production or modulation of ERK activity will be potentially effective medicines for PD aiming at mitigation or prevention of DA neuron degeneration and reduction of LID. Experiments with apocynin have indeed shown that this principle can be used to prevent the assembly and activation of the ROS generating NADPH oxidase in the MPTP marmoset model.

The studies described herein used the solid state formulations of 3β,7β-Bis-(trimethylsiloxy)-5-androsten-17-one that are described in U.S. Pat. No. 8,252,947.

### Example 2. Treatment of Motor Symptoms in the MPTP Model of Parkinson's Disease in Marmoset Monkeys

3β,7β-Bis-(trimethylsiloxy)-5-androsten-17-one (17-EAT) has a unique mechanism of action that targets ERK and decreases inflammatory signaling by inhibiting ERK and NFkB activation in specific inflammatory signaling scaffolds that drive pathological inflammation cascades. All experimental evidence to date suggests that 17-EAT does not inhibit ERK and NFkB homeostatic functions. 17-EAT is blood-brain barrier permeable and decreases expression of TNF, IL-1b, IL-6, and other inflammatory cytokines. 17-EAT is active in rodent models of systemic inflammation (type 2 diabetes, COPD, rheumatoid arthritis), and has anti-neuroinflammatory or neuroprotective activity in rodent models of Parkinson's disease, experimental autoimmune encephalomyelitis, optic neuritis, and glaucoma.

The 1-methyl-4-phenyl-1,2,3,6-tetrahydropyridine (MPTP) model that was used is capable of reducing striatal dopamine levels to 5% of normal controls. The MPTP model was used to produce Parkinson's-like disease symptoms in three sequential independent cohorts of six marmosets monkeys (Callithrix jacchus, approximately 0.3-0.5 kg, 2-5 years) which were then observed for 3 weeks to confirm the absence of parkinsonian behavior and motor abnormalities. A single dose of 17-EAT or amantadine was administered at the beginning of Week 4 to confirm the absence of behavioral side effects. All subjects received subcutaneous injections of 1-2 mg/kg MPTP on Days 3, 4, and 5 of Week 5 and Day 1 and 3 of Week 6 for a total of 6.5 mg/kg MPTP and were observed through the end of Week 7 without further treatment, then randomized for treatment. All MPTP treated subjects displayed obvious parkinsonian behaviors.

Oral administration of treatment of vehicle, amantadine, or 17-EAT formulated in acacia syrup began at the start of Week 8. Vehicle controls received acacia syrup only. Amantadine HCl (Sigma Aldrich, A1260-SG) was administered daily at a dose of 1 mg/kg. 17-EAT was administered daily at a dose of 30 mg/kg. In Weeks 8 and 9 test compound activity against parkinsonian behavior was observed several times daily for effects on clinical signs (lack of grooming, apathy, immobility, muscle rigidity, and tremor activity) and given an immobility score on a 0-4 scale, with 0 being normal and 4 being the most severe score. Ratings were performed by study personnel blind to treatment. Subjects were observed for parkinsonian signs as in Weeks 8 and 9 and for expression of LID.

In Weeks 10 and through the middle of Week 11, all subjects received single escalating doses of levodopa (5, 7.5, 10, and 12.5 mg/kg) in combination with continuing daily vehicle, amantadine, or 17-EAT with one to two days of observation between levodopa doses. In Weeks 11-13, LID was induced in all subjects with 12.5 mg/kg levodopa twice daily, while continuing vehicle, amantadine, or 17-EAT treatments.

The severity of LID was measured by abnormal involuntary movements score (AIMS) adapted to the primate PD model. The items include assessment of extremity and trunk movements, facial expression, and movement of the lips, peri-oral area, tongue, and jaw. These symptoms were scored on a scale from 0 (normal), 1 (extreme normal), 2 (mild), 3 (moderate) to 4 (severe). Measurement of parkinsonian behaviors continued as previously described. In Week 14 therapies were continued without levodopa, and the subjects were euthanized (one subject from each of the three groups on Days 1, 2, and 3) 1 hour after 17-EAT treatment. The subjects were exsanguinated under deep anaesthesia with Alphaxan (18 mg/kg i.m.). Immediately thereafter, the subjects were euthanized with EUTHASOL (pentobarbital natrium, i.c.). The brain of each subject was removed and frozen for immunohistochemical analyses. SNpc was analyzed for the presence of DA positive neurons with tyrosine hydroxylase immune reactive (TH-IR) staining.

Mean immobility scores of the subjects treated with vehicle, amantadine, and 17-EAT prior to treatment (Week 7) were 2.71, 2.59, and 2.72, respectively, and 3.0, 2.74, and 2.26, respectively, in the first week of monotherapy treatment (Week 8) as shown in Table 1. Mean immobility scores for 17-EAT monotherapy at Weeks 8 and 9 were 2.26 and 2.10, respectively, and significantly lower than monotherapy treatment with either the vehicle (3.0 and 2.8), or amantadine (2.74 and 2.72).

TABLE 1

| | | | | P value |
|---|---|---|---|---|
| | | Amantadine | 17-EAT | 17-EAT vs |
| Week | Vehicle | (P*) | (P*) | amantadine |
| 7 | 2.71 | 2.59 | 2.72 | 0.433 |
| | | (0.58) | (0.86) | |
| 8 | 3.0 | 2.74 | 2.26 | 0.0048 |
| | | (0.104) | (2.8E−06) | |
| 9 | 2.8 | 2.72 | 2.10 | 0.0044 |
| | | (0.750) | (8.8E−04) | |

*denotes P values between treatment and vehicle

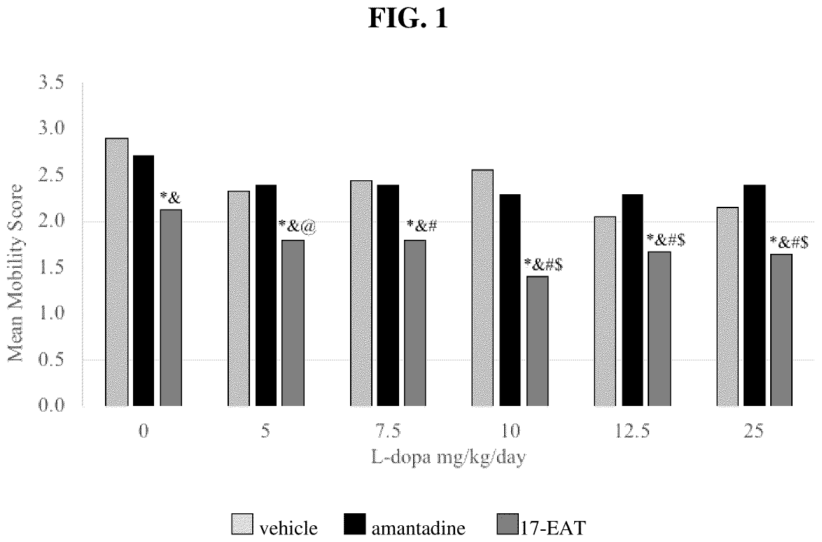

Mean immobility scores in Week 9 of subjects treated with 17-EAT administered as 12.5 mg/kg twice daily in combination with a levodopa dosage of 5, 7.5, 10, 12.5 or 25 mg/kg were improved compared to 17-EAT monotherapy, as shown in FIG. 1. The addition of levodopa improved immobility scores compared to monotherapy evaluated in Weeks 8 and 9 (data shown for Week 9 values).

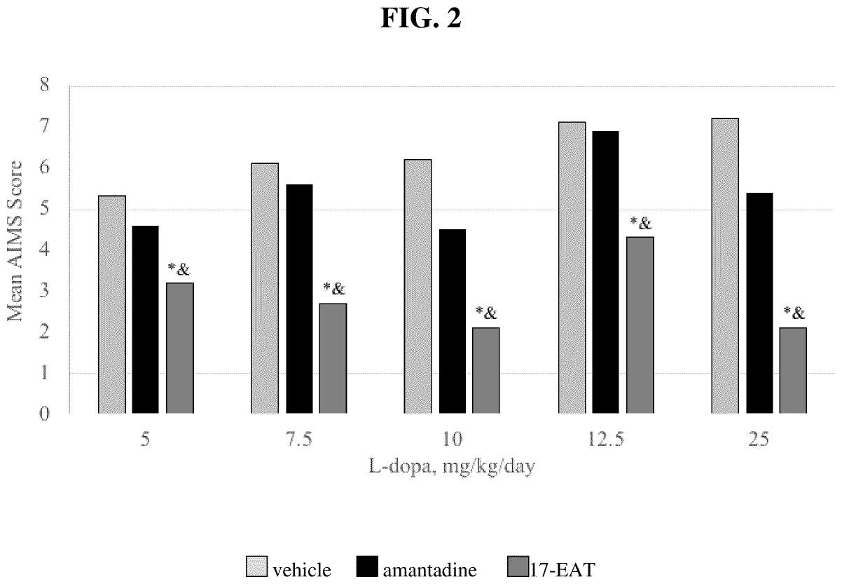

Levodopa dosage of 12.5 mg/kg BID administered twice daily induced LID expression in all subjects. Abnormal involuntary movements scores (AIMS) in Week 12 of subjects treated with vehicle, amantadine, and 17-EAT in combination with a levodopa dosage of 5, 7.5, 10, 12.5 or 25 mg/kg were improved compared to monotherapy, as shown in FIG. 2. AIMS in Week 12 of subjects treated with vehicle, amantadine, and 17-EAT in combination with a levodopa dosage of 10 mg/kg were 6.2, 4.5, and 2.1, respectively.

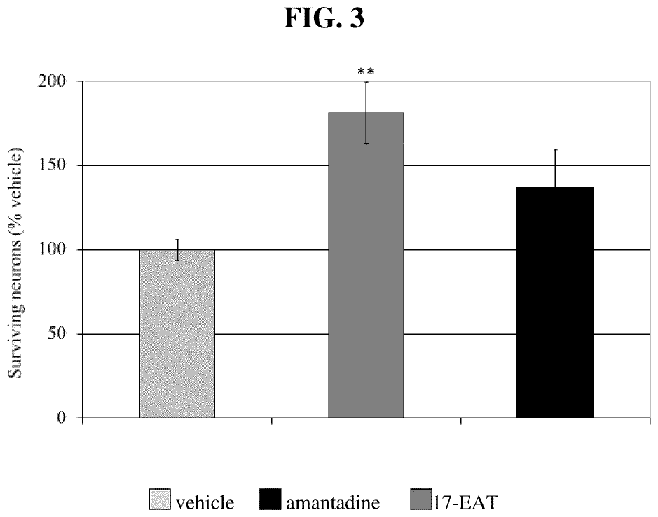

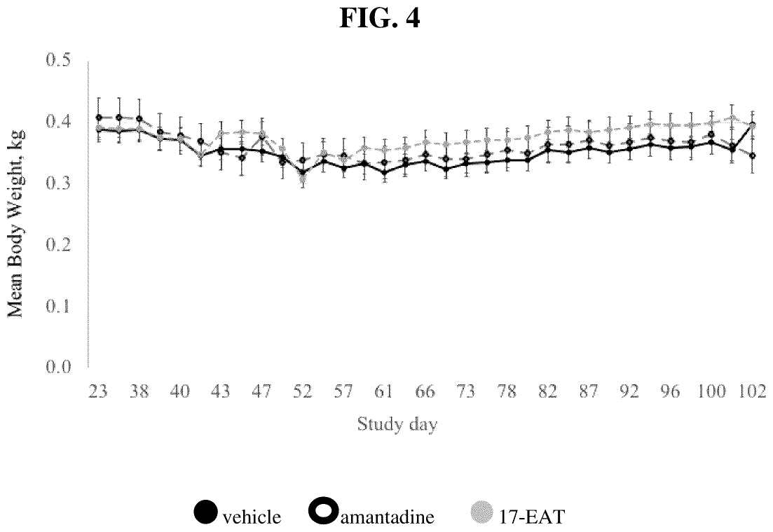

In Week 14, all subjects were sacrificed and tissue samples from the substantia nigra were evaluated to measure the number of surviving tyrosine hydroxylase positive neurons. The increase in number of surviving TH+ neurons for subjects treated with amantadine and 17-EAT was 40% and 75%, as shown in FIG. 3. The subjects that were treated with 17-EAT maintained a higher mean body weight compared to either vehicle or amantadine, as shown in FIG. 4.

### Example 3. Delay of Need to Start Dopamine Agonist Therapy and Decrease Motor Complications

Based on the inventor's clinical experience, the following results are projected using controlled studies.

A cohort of 40 patients having Parkinson's disease between the ages of 35 and 80 years of age is identified by a physician as naïve to dopamine agonist therapy and with motor symptoms that have recently progressed to a level that requires therapy in order to be decreased. The patients are divided into one group of patients (n=20; “SSF”) that receives the solid state formulation orally twice daily and a second group of patients (n=20; “LVD”) that receives L-dopa treatment as needed to control motor symptoms. Patient symptoms are evaluated at three-month intervals. After two years, motor symptoms are satisfactorily controlled in both groups. After four years, motor symptoms are satisfactorily controlled in the SSF group and motor symptoms are not completely controlled and accompanied by motor complications that are observed in approximately 20% of the LVD group. After six years, control of motor symptoms in the SSF group require the addition of low dose L-dopa to be satisfactory controlled; patients in the LVD group have progressively greater and statistically significant greater increase of motor symptoms and motor complications and/or dyskinesia.

### Example 4. Delay of Motor Complications

Based on the inventor's clinical experience, the following results are projected using controlled studies.

A cohort of 40 patients having Parkinson's disease between the ages of 35 and 80 years of age is identified by a physician. A detailed examination report for each patent is prepared, complete with an indication of symptoms and their severity. Symptoms common to the patients include trembling in hands, arms, legs, jaw, or head; stiffness of the limbs and trunk; slowness of movement; and impaired balance and/or coordination. This report establishes a patient baseline. The experimental group patients (n=20; “EXPT1”) receive the solid state formulation orally twice daily and L-dopa as needed daily to optimize treatment of disease symptoms. The control group patients (n=20; “CONT”) receive a placebo twice daily and L-dopa as needed daily to optimize treatment of disease symptoms. The study is conducted for one year wherein patient outcomes are measured by the physician at 1 month intervals. Patients receiving the EXPT1 and the CONT report improvement in each symptom after 1 to 4 months. The study is conducted for an additional 20 years during which patient outcomes are measured by the physician. A first half of the patients receiving the EXPT1 report no return of the original symptoms and no development of new symptoms over the course of the study. A second half of the patients receiving the EXPT1 report no return of the original symptoms and development of motor symptoms including motor fluctuations and motor complications including wearing off, freezing of gait, and acute akinesia after 5 to 16 years. A portion of patients receiving the CONT report gradual development of motor symptoms including motor fluctuations and motor complications including wearing off, freezing of gait, and acute akinesia after 2 to 4 years. The difference between motor symptoms in the EXPT1 group versus the CONT group is statistically significant.

### Example 5. Delay of Dyskinesia

Based on the inventor's clinical experience, the following results are projected using controlled studies.

A cohort of 40 patients having Parkinson's disease between the ages of 35 and 80 years of age is identified by a physician. A detailed examination report for each patent is prepared, complete with an indication of symptoms and their severity. Symptoms common to the patients include trembling in hands, arms, legs, jaw, or head; stiffness of the limbs and trunk; slowness of movement; and impaired balance and/or coordination, but without significant motor complications or L-dopa induced dyskinesia. This report establishes a patient baseline. The experimental group patients (n=20; “EXPT1”) receive the solid state formulation orally twice daily and L-dopa as needed daily. The control group patients (n=20; “CONT”) receive a placebo orally twice daily and L-dopa as needed daily. The study is conducted for five years wherein patient outcomes are measured by the physician at 3 month intervals. Patients receiving the EXPT1 and the CONT report improvement in each symptom after 1 to 4 months. The study is conducted for an additional 20 years during which patient outcomes are measured by the physician. A first half of the patients receiving the EXPT1 report no return of the original symptoms and no development of new symptoms over the course of the study. A second half of the patients receiving the EXPT1 report no return of the original symptoms and development of dyskinesia including chorea, dystonia, ballism, and myoclonus after 5 to 16 years. Patients receiving the CONT report no return of the original symptoms and development of dyskinesia including chorea, dystonia, ballism, and myoclonus after 2 to 4 years. The difference between dyskinesia in the EXPT1 group versus the CONT group is statistically significant.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description

All publications, patent applications, issued patents, and other documents (for example, journals, articles and/or textbooks) referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims, along with the full scope of equivalents to which such claims are entitled.

While the subject matter has been particularly shown and described with reference to a preferred embodiment and various alternate embodiments, it will be understood by persons skilled in the relevant art that various changes in form and details can be made therein without departing from the spirit and scope of the present disclosure.

What is claimed is:

1. A method to treat a neurodegenerative condition, the method comprising administering to a patient in need thereof an effective amount of a pharmaceutical composition comprising 17α-ethynylandrost-5-ene-3β,7β,17β-triol and at least one pharmaceutically acceptable excipient.

2. The method of claim 1, wherein the neurodegenerative condition is Parkinson's disease.

3. The method of any one of claims 1 to 2, wherein the method further comprises administering at least one additional medicament to the patient.

4. The method of claim 3, wherein the additional medicament comprises at least one dopamine agonist.

5. The method of any one of claims 3 to 4, wherein the additional medicament comprises at least one dopamine precursor.

6. The method of any one of claims 3 to 5, wherein the additional medicament comprises L-dopa.

7. The method of any one of claims 3 to 6, wherein the additional medicament is administered at a delay time after a first administration of the composition.

8. The method of claim 7, wherein the delay time is equal to or greater than 2 years.

9. The method of any one of claims 1 to 8, wherein at least one motor symptom and/or at least one motor complication develops in the patient.

10. The method of claim 9, wherein the motor symptom is selected from tremor and/or shaking in the extremities, slowed movement (bradykinesia), muscle stiffness, rigidity, immobility (freezing), muscle cramps, impaired posture and/or balance, falls, dizziness, loss of automatic movements such as blinking or smiling, changes in speech and/or writing, motor fluctuations, dystonia, and any combination of the foregoing.

11. The method of claim 9, wherein the motor complication is selected from wearing off, dose failure, beginning of dose worsening, end-of-dose rebound, unpredictable off-periods, freezing of gait, on-period failure, acute akinesia, dyskinesia, and any combination of the foregoing.

12. The method of any one of claims 9 to 11, wherein the motor symptom and/or the motor complication develops at a time equal to or greater than 2 years after the additional medicament is administered.

13. The method of any one of claims 1 to 12, wherein the 17α-ethynylandrost-5-ene-3β,7β,17β-triol is a solid state form of 17α-ethynylandrost-5-ene-3β,7β,17β-triol.

14. The method of claim 13, wherein the solid state form of 17α-ethynylandrost-5-ene-3β,7β,17β-triol is crystalline solvate of 17α-ethynylandrost-5-ene-3β,7β,17β-triol.

15. The method of claim 14, wherein the crystalline solvate is crystalline methanolate 17α-ethynylandrost-5-ene-3β,7β,17β-triol.

16. The method of claim 14, wherein the crystalline solvate is crystalline ethanolate 17α-ethynylandrost-5-ene-3β,7β,17β-triol.

17. The method of claim 14, wherein the crystalline solvate is crystalline hydrate 17α-ethynylandrost-5-ene-3β,7β,17β-triol.

18. The method of claim 14, wherein the crystalline solvate is Form III 17α-ethynylandrost-5-ene-3β,7β,17β-triol.

19. The method of claim 14, wherein the crystalline solvate is Form IV 17α-ethynylandrost-5-ene-3β,7β,17β-triol.

20. The method of claim 14, wherein the crystalline solvate is Form V 17α-ethynylandrost-5-ene-3β,7β,17β-triol.

21. The method of claim 13, wherein the solid state form of 17α-ethynylandrost-5-ene-3β,7β,17β-triol is amorphous 17α-ethynylandrost-5-ene-3β,7β,17β-triol.

22. The method of any one of claims 1 to 21, wherein the pharmaceutical composition contains less than about 3% by weight of impurities.

23. The method of any one of claims 1 to 22, wherein the patient is a human or mammal.

24. A pharmaceutical composition comprising 17α-ethynylandrost-5-ene-3β,7β,17β-triol and at least one pharmaceutically acceptable excipient for use in treating a neurodegenerative condition.

25. The use of a pharmaceutical composition comprising 17α-ethynylandrost-5-ene-3β,7β,17β-triol and at least one pharmaceutically acceptable excipient for treating a neurodegenerative condition.

26. The use of 17α-ethynylandrost-5-ene-3β,7β,17β-triol in the manufacture of a medicament for treating a neurodegenerative condition, wherein the medicament comprises at least one pharmaceutically acceptable excipient.

27. The pharmaceutical composition of claim 24 or the use of claim 25 or 26, wherein the neurodegenerative condition is Parkinson's disease.

28. The pharmaceutical composition of claims 24 or 27, or the use of claims 25-27, wherein the composition, or the use, comprises at least one additional medicament.

29. The pharmaceutical composition of claims 24 or 27-28 or the use of claims 25-28, wherein the additional medicament comprises L-dopa.

30. The pharmaceutical composition of claims 24 or 27-29 or the use of claims 25-29, wherein the additional medicament is used at a delay time after use of the pharmaceutical composition begins.

31. The pharmaceutical composition of claims 24 or 27-30 or the use of claims 25-30, wherein the delay time is equal to or greater than 2 years.

32. The pharmaceutical composition of claims 24 or 27-31 or the use of claims 25-31, wherein development of at least one motor symptom and/or at least one motor complication occurs.

33. The pharmaceutical composition of claims 24 or 27-32 or the use of claims 25-32, wherein the motor symptom is selected from tremor and/or shaking in the extremities, slowed movement (bradykinesia), muscle stiffness, rigidity, immobility (freezing), muscle cramps, impaired posture and/or balance, falls, dizziness, loss of automatic movements such as blinking or smiling, changes in speech and/or writing, motor fluctuations, dystonia, and any combination of the foregoing.

34. The pharmaceutical composition of claims 24 or 27-33 or the use of claims 25-33, wherein the motor complication is selected from wearing off, dose failure, beginning of dose worsening, end-of-dose rebound, unpredictable off-periods, freezing of gait, on-period failure, acute akinesia, dyskinesia and any combination of the foregoing.

35. The pharmaceutical composition of claims 24 or 27-34 or the use of claims 25-34, wherein the motor symptom and/or the motor complication develops at a time equal to or greater than 2 years after the use of the pharmaceutical composition begins.

36. The pharmaceutical composition of claims 24 or 27-35 or the use of claims 25-35, wherein the motor symptom and/or the motor complication develops at a time equal to or greater than 2 years after the use of the additional medicament begins.

37. The pharmaceutical composition of claims 24 or 27-36 or the use of claims 25-36, wherein the 17α-ethynylandrost-5-ene-3β,7β,17β-triol is a solid state form of 17α-ethynylandrost-5-ene-3β,7β,17β-triol.

38. The pharmaceutical composition of claims 24 or 27-37 or the use of claims 25-37, wherein the solid state form is a crystalline solvate of 17α-ethynylandrost-5-ene-3β,7β,17β-triol.

39. The pharmaceutical composition of claims 24 or 27-38 or the use of claims 25-38, wherein the crystalline solvate is crystalline methanolate 17α-ethynylandrost-5-ene-3β,7β,17β-triol.

40. The pharmaceutical composition of claims 24 or 27-39 or the use of claims 25-39, wherein the crystalline solvate is crystalline ethanolate 17α-ethynylandrost-5-ene-3β,7β,17β-triol.

41. The pharmaceutical composition of claims 24 or 27-40 or the use of claims 25-40, wherein the crystalline solvate is crystalline hydrate 17α-ethynylandrost-5-ene-3β,7β,17β-triol.

42. The pharmaceutical composition of claims 24 or 27-41 or the use of claims 25-41, wherein the crystalline solvate is Form III 17α-ethynylandrost-5-ene-3β,7β,17β-triol.

43. The pharmaceutical composition of claims 24 or 27-42 or the use of claims 25-42, wherein the crystalline solvate is Form IV 17α-ethynylandrost-5-ene-3β,7β,17β-triol.

44. The pharmaceutical composition of claims 24 or 27-43 or the use of claims 25-43, wherein the crystalline solvate is Form V 17α-ethynylandrost-5-ene-3β,7β,17β-triol.

45. The pharmaceutical composition of claims 24 or 27-44 or the use of claims 25-44, wherein the solid state form is amorphous 17α-ethynylandrost-5-ene-3β,7β,17β-triol.
